(19) United States
(12) Patent Application Publication
(10) Pub. No.: US 2015/0064114 A1
(43) Pub. Date: Mar. 5, 2015

(54) LIPOSOME INCLUDING HYDROPHOBIC MATERIAL AND IMAGING AGENT AND USE OF THE LIPOSOME

(71) Applicant: Samsung Electronics Co., Ltd., Suwon-si (KR)

(72) Inventors: Sun Min PARK, Daegu (KR); Kitae PARK, Yongin-si (KR); Hyun Ryoung KIM, Guri-si (KR); Eun Sung PARK, Seongnam-si (KR)

(21) Appl. No.: 14/301,504

(22) Filed: Jun. 11, 2014

(30) Foreign Application Priority Data
Sep. 3, 2013 (KR) 10-2013-0105696

(51) Int. Cl. A61K 9/127; A61K 31/44; A61M 37/00; A61K 31/28

(52) U.S. Cl. CPC A61K 9/127; A61K 31/28; A61K 31/44; A61M 37/0092
USPC 424/9.51; 424/450; 514/492; 514/346; 600/431

(57) ABSTRACT

A liposome comprising a lipid bilayer, a hydrophobic active ingredient, and an imaging agent, a pharmaceutical composition including the liposome, and a method of delivering an active ingredient to a target site in the body of a subject using the liposome.

### RELATED APPLICATION

This application claims the benefit of Korean Patent Application No. 10-2013-0105696, filed on Sep. 3, 2013, in the Korean Intellectual Property Office, the disclosure of which is hereby incorporated by reference.

### BACKGROUND

1. Field

The present disclosure relates to liposomes comprising lipid bilayers, hydrophobic active ingredients, and imaging agents, pharmaceutical compositions including the liposomes, and methods of delivering the hydrophobic active ingredient to a target site in the body of a subject by using the liposomes.

2. Description of the Related Art

Liposomes consist of at least one lipid bilayer membrane enclosing an aqueous internal compartment. Liposomes may be characterized by membrane type and by size. Small unilamellar vesicles (SUVs) may have a single membrane and have a diameter in a range from about 20 nm to about 50 nm. Large unilamellar vesicles (LUBs) may have a diameter of about 50 nm or greater. Oligolamellar large vesicles and multilamellar vesicles may have multiple, usually concentric, membrane layers and have a diameter of about 100 nm or greater. Liposomes with several non-concentric membranes, i.e., several smaller vesicles contained within in a large vesicle, are known as multivesicular vesicles.

Liposomes may be formulated to carry therapeutic agents, drugs or other active ingredients that are either contained within the aqueous interior space (a water-soluble active ingredient) or partitioned into the lipid bilayer (water-insoluble active ingredient). In addition, a hydrophobic material, such as cholesterol, may be contained in a micelle, which is an aggregate comprising a hydrophobic interior and a hydrophilic exterior. A micelle comprising a hydrophobic material may be contained within the aqueous interior space of a liposome.

The delivery of hydrophobic drugs may be performed by emulsion, through the use of co-solvents, and by micelles. With respect to liposomes, hydrophobic drugs and solubilizers thereof may be encapsulated within a liposome interior. However, the addition of solubilizers to liposomes that comprise one or more drugs creates a risk that the liposomes may not encapsulate the drugs in a sufficient amount or that the solubilizers will decrease stability of the liposomes themselves. In addition, the only area that can encapsulate drugs or physiologically active ingredients is an interior space of the liposome. Issues with drug delivery may arise, for example, when a liposome comprises two or more drugs. For example, the two or more drugs may undesirably react with one another before they can be effectively delivered to a target site. Additionally, drugs may interact with the liposome itself, thus interfering with the ability to control their release. As an example, when an anticancer drug such as paclitaxel is introduced into the lipid bilayer membrane of a liposome, the liposome may be degraded and the drug contained therein may be released slowly. Thus, there is a limit in regard to performance and functionality of liposomes as drug delivery vehicles.

Accordingly, there remains a need for liposomes that do not have a limit on the amount of hydrophobic agents they may comprise, as well as liposomes that are capable of controlled drug release.

### SUMMARY

Provided is a liposome comprising a lipid bilayer, hydrophobic active ingredient, and imaging agent, wherein the hydrophobic active ingredient is in the lipid bilayer and the imaging agent is contained in an interior space of the liposome.

Additionally provided is a pharmaceutical composition comprising the liposome. Further provided is a method for delivering an active ingredient to a target site in the body of a subject comprising: administering a liposome comprising a hydrophobic active ingredient and an imaging agent to a subject; and applying ultrasound to a target site of the subject to release the hydrophobic ingredient and the imaging agent from the liposome.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic diagram illustrating the functionality of ultrasound-sensitive liposomes according to an aspect of the present invention (□: hydrophobic drug, ▪: imaging agent);

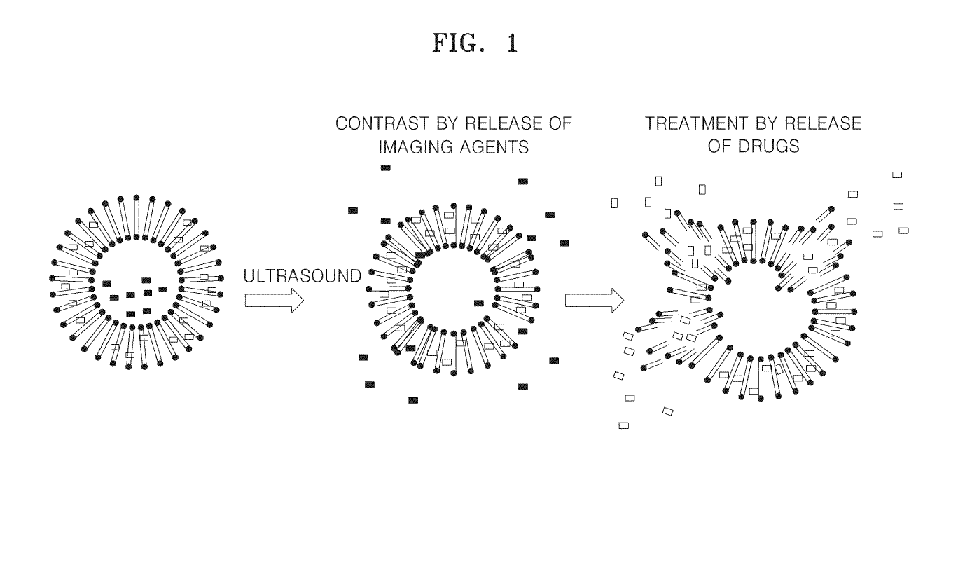

FIG. 2 is a graph showing diameters of liposomes (X-axis) as measured by dynamic light scattering;

FIG. 3 is a transmission electron microscope (TEM) image of liposomes prepared according to an aspect of the present invention;

FIG. 4 is a graph showing cell viability (%) (Y-axis) of breast cancer cells exposed to various concentrations of sorafenib and sorafenib-containing liposomes with and without exposure to general ultrasound (: sorafenib, ◯: sorafenib-containing liposome, ▾: sorafenib-containing liposome+ultrasound);

FIG. 5A is a graph showing sizes of liposomes (Y-axis) after exposure to high-intensity focused ultrasound (HIFU);

FIG. 5B is a graph showing amount of hydrophobic materials released from liposomes exposed to HIFU as measured by fluorescent intensity (Y-axis) (□: before exposure to HIFU, ▪: after exposure to HIFU);

FIG. 6A is a graph showing average sizes (Y-axis) of liposomes comprising different combinations of phospholipids before and after exposure to HIFU;

FIG. 6B is a graph showing the average sizes (Y-axis) of 95% or more of liposomes comprising different combinations of phospholipids before and after exposure to HIFU; and

FIG. 6C is a graph showing the ratios of hydrophobic materials (%) (X-axis) present in liposomes comprising different combinations of phospholipids before and after exposure to HIFU; and

FIG. 7 is a magnetic resonance image of liposomes before and after exposure to HIFU.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. Expressions such as “at least one of,” when preceding a list of elements, modify the entire list of elements and do not modify the individual elements of the list.

According to an aspect of the present invention, provided is a liposome comprising a lipid bilayer, a hydrophobic active ingredient, and an imaging agent, wherein the hydrophobic active ingredient is in the lipid bilayer and the imaging agent is contained in an interior space of the liposome.

The term “liposome” as used herein refers to an artificially prepared vesicle composed of a lipid bilayer. A liposome may be classified as a unilamellar vesicle or a multivesicular vesicle.

The liposome may be an ultrasound-sensitive liposome. The term “ultrasound” as used herein refers to a wave with a frequency greater than an audio frequency ranging from about 16 Hz to about 20 Hz. Ultra-sound sensitive liposomes refer to liposomes that are capable of releasing their contents (e.g., hydrophobic active ingredient and imaging agent) upon exposure to one or more types of ultrasound. The liposome may be sensitive to high-intensity focused ultrasound (HIFU). HIFU involves the use of high-intensity ultrasound energies in one place to create a concentrated focus. Imaging technologies may guide the administration HIFU, and thus HIFU may be classified, e.g., as ultrasound-guided HIFU or magnetic resonance imaging (MRI)-guided HIFU. HIFU may have a frequency, for example, in a range of about 20 kHz to about 2.0 MHz, about 40 kHz to about 2.0 MHz, about 60 kHz to about 2.0 MHz, about 80 kHz to about 2.0 MHz, about 100 kHz to about 2.0 MHz, about 150 kHz to about 2.0 MHz, about 200 kHz to about 2.0 MHz, about 250 kHz to about 2.0 MHz, about 300 kHz to about 2.0 MHz, about 350 kHz to about 2.0 MHz, about 400 kHz to about 2.0 MHz, about 450 kHz to about 2.0 MHz, about 500 kHz to about 2.0 MHz, about 550 kHz to about 2.0 MHz, about 600 kHz to about 2.0 MHz, about 650 kHz to about 2.0 MHz, about 700 kHz to about 2.0 MHz, about 750 kHz to about 2.0 MHz, about 800 kHz to about 2.0 MHz, about 850 kHz to about 2.0 MHz, about 900 kHz to about 2.0 MHz, about 950 kHz to about 2.0 MHz, about 1.0 MHz to about 2.0 MHz, about 1.1 MHz to about 1.9 MHz, about 1.2 MHz to about 1.8 MHz, about 1.3 MHz to about 1.7 MHz, or about 1.4 MHz to about 1.6 MHz. In one embodiment, HIFU may have a frequency of about 1.5 MHz.

The term “lipid bilayer” as used herein refers to a membrane made of two layers of lipid molecules. The lipid bilayer may have a similar thickness as that of a naturally existing bilayer, such as a cell membrane, a nuclear membrane, and a virus envelope. For example, the lipid bilayer may have a thickness of about 10 nm or less, for example, in a range of about 1 nm to about 9 nm, about 2 nm to about 8 nm, about 2 nm to about 6 nm, about 2 nm to about 4 nm, or about 2.5 nm to about 3.5 nm. The lipid bilayer is a barrier that retains ions, proteins, and other molecules while also preventing them from diffusing into undesirable areas. The “lipid molecules” forming the lipid bilayer may comprise a molecule including a hydrophilic head and a hydrophobic tail. The lipid molecule may comprise from about 14 to about 50 carbon atoms.

Examples of the lipid molecules which may form a lipid bilayer include phospholipids, lipids conjugated to polyethylene glycol (PEG), cholesterol, or any combination thereof.

The phospholipid is a compound lipid containing phosphate ester within a molecule, and is a main component of biological membranes, such as cell membranes, endoplasmic reticulum, mitochondria, and myelin sheath around nerve fibers. The phospholipid includes a hydrophilic head and two hydrophobic tails. When the phospholipids are exposed to water, they arrange themselves into a two-layered sheet (a lipid bilayer) with all of their tails pointing toward the center of the sheet. The center of this lipid bilayer contains almost no water and also excludes molecules such as sugars or salts that dissolve in water but not in oil. The phospholipids with certain head groups may alter the surface chemistry of a lipid bilayer. Also, lipid tails may affect membrane properties by, for example, determining the phase of the lipid bilayer. The lipid bilayer may adopt a solid gel phase state at lower temperatures, but undergo phase transition to a fluid state at higher temperatures. The packing of lipids within the lipid bilayer may also affect its mechanical properties, including its resistance to stretching and bending. Biological membranes may include several types of lipids other than phospholipids.

Examples of phospholipids include phosphatidic acid, phosphatidylethanolamine, phosphatidylcholine, phosphatidylserine, phosphatidylinositol, phosphosphingolipid, or any combination thereof. Phosphatidylcholine (PC) may include choline as a head group and glycerophosphoric acid as a tail, wherein glycerophosphoric acid may be saturated fatty acid or unsaturated fatty acid and have 14 to 50 carbon atoms. Examples of PC include 1,2-dipalmitoyl-sn-glycero-3-phosphocholine (DPPC), 1,2-distearoyl-sn-glycero-3-phosphocholine (DSPC), egg PC, soy bean PC, and any combination thereof. In the phospholipid bilayer, DPPC and egg PC may be contained in a ratio in a range of 4:1 to 1:4, 3:1 to 1:3, or 2:1 to 1:2. For example, the ratio of DPPC and egg PC may be 1:1.

The lipid conjugated to PEG may be phosphatidylethanolamine (PE)-PEG. The PE may comprise saturated fatty acid, unsaturated fatty acid, mixed acyl chain, lysophosphatidylethanolamine, or any combination thereof. The lipid conjugated with PEG may comprise for example 1,2-distearoylphosphatidylethanolamine-methyl-polyethylene glycol (DSPE-PEG).

The term “cholesterol” as used herein refers to any suitable steroid compounds. Cholesterol, as used herein, also refers to a cholesterol derivative, and examples thereof include sitosterol, ergosterol, stigmasterol, 4,22-stigmastadiene-3-on, stigmasterol acetate, lanosterol, cycloartenol, or any combination thereof. Cholesterol may enhance fluidity of a lipid bilayer and lower the permeability of the lipid bilayer.

The term “hydrophobic” as used herein refers to properties that a material possesses that do not allow it to easily combine with a water molecule or do not allow it to easily dissolve in water, or refers to non-polar properties of the material. The term “hydrophobic” as used herein may be used interchangeably with the term “lipophilic”. Hydrophobic materials may be classified according to water solubility thereof at 25° C. and 1 atm (e.g., slightly soluble in a range of about 1 mg/ml to about 10 mg/ml, very slightly soluble in a range of about 0.1 mg/ml to about 1 mg/ml, and substantially insoluble at the level of about 0.1 mg/ml or less).

The term “active agent” as used herein refers to a material of biologically active pharmaceutical drugs or herbicides. Examples of the active ingredient include a compound, a protein, a peptide, a nucleic acid, a nanoparticle, or any combination thereof.

Examples of the active ingredient include anticancer drugs, anti-angiogenesis inhibitors, anti-inflammatory drugs, analgesics, antiarthritics, sedatives, antidepressants, antipsychotics, tranquilizers, antianxiety drugs, narcotic antagonists, anti-Parkinson's disease drugs, cholinergic agents, immunosuppressive agents, antiviral agents, antibiotics, appetite suppressants, anticholinergics, antihistamines, anti-migraine drugs, hormones, vasodilators, birth control pills, antithrombotic agents, diuretics, antihypertensives, cardiovascular drugs, wrinkle-diminishing agent, inhibitors of skin aging, skin whitening agent, or any combination thereof.

The hydrophobic active ingredient may comprise a hydrophobic drug, and examples thereof include sorafenib, paclitaxel, cyclosporine A, amphothericin B, indinavir, or any combination thereof. Sorafenib may be used for the treatment of kidney cancer and liver cancer. Paclitaxel may be used for the treatment of ovarian cancer, breast cancer, or lung cancer. Cyclosporine A may be used as an immunosuppressive agent. Amphothericin B may be used as a polyene antibiotic. Indinavir may be used as a protease inhibitor. The hydrophobic active ingredient may comprise a steroid-based substance, and examples thereof include glucocorticoid, taxane-based drugs, cyclic peptide-based drugs (e.g., cyclosporine A), indinavir, amphotericin B, or any combination thereof. Examples of the hydrophobic glucocoriticoid include dexamethasone, triamcinolone, beclomethasone diproprionate, triamcinolone acetonide, triamcinolone diacetate, bethamethasone diproprionate, testosterone, budesonide, 17α-ethinylestradiol, levonorgestrel, fluticasone proprionate, or any combination thereof.

The term “imaging agent (or a contrast medium)” as used herein refers to a substance to enhance contrast of an image showing tissues or blood vessels clearly at the time of examination such as magnetic resonance imaging (MRI) and computed tomography (CT) by artificially increasing X-ray absorption differences of each tissue. The imaging agent may be classified as a positive imaging agent and a negative imaging agent. The negative imaging agent is more permeable to X-ray than surrounding tissues to represent an image. For example, the positive imaging agent may be iodine-containing imaging agent or barium sulfate, and the negative imaging agent may be air, gas, or carbon dioxide. The imaging agent may include transition elements or chelate complexes of transition elements. Examples of the transition elements include lanthanum (La), praseodymium (Pr), neodymium (Nd), gadolinium (Gd), terbium (Tb), manganese (Mn), zinc (Zn), iron (Fe), scandium (Sc), titanium (Ti), vanadium (V), zinc (Zn), yttrium (Y), zirconium (Zr), niobium (Nb), molybdenum (Mo), palladium (Pd), silver (Ag), cadmium (Cd), tungsten (W), or rhenium (Re). The transition elements may be in the form of ions. For example, gadolinium (having a symbol of Gd and an atomic number of 64) may be Gd³⁺. The chelate complex of Gd may include gadoteric acid, gadodiamide, gadobenic acid, gadopentetic acid, gadoteridol, gadoversetamide, gadoxetatic acid, gadobutrol, or any combination thereof.

The hydrophobic active ingredient and the imaging agent may be positioned in different areas in the liposome. For example, the hydrophobic active ingredient may be in the lipid bilayer of the liposome, and the imaging agent may be contained in the interior space of the liposome. The interior space of the liposome may comprise a liposome interior with respect to the structure of the lipid bilayer.

The liposome may further include a hydrophilic active ingredient. The term “hydrophilic” as used herein refers to properties of a material that allows the material to easily combine with a water molecule or easily dissolve in water, or refers to polar properties of the material. Examples of the hydrophilic active ingredient include methotrexate, doxorubicin, epirubicin, daunorubicin, vincristine, vinblastine, etoposide, ellipticine, camptothecin, docetaxel, cisplatin, prednisone, methyl-prednisone, ibuprofen, idarubicin, valrubicin, mitoxantrone, ampicillin, streptomycin, penicillin, or any combination thereof.

The liposome may have a diameter in a range of about 50 nm to about 500 nm, for example, about 50 nm to about 400 nm, about 50 nm to about 300 nm, about 50 nm to about 200 nm, or about 50 nm to about 150 nm. The liposome may be a unilamellar vesicle (UV) or a multivesicular vesicle.

According to another aspect of the present invention, provided is a pharmaceutical composition for delivering a hydrophobic active ingredient to a subject that includes a liposome comprising a lipid bilayer, a hydrophobic active ingredient, and an imaging agent, wherein the hydrophobic active ingredient is in the lipid bilayer (i.e., included within the lipid or hydrophobic portion of the bilayer itself, rather than the interior space of the liposome) and the imaging agent is contained in an interior space of the liposome.

The lipid bilayer, the hydrophobic active ingredient, the imaging agent, and the liposome may comprise characteristics as previously described, above.

The pharmaceutical composition may further include a pharmaceutically acceptable carrier or a diluent. The pharmaceutically acceptable carrier or diluent may be well known in the art. Examples of the pharmaceutically acceptable carrier or diluents include lactose, dextrose, sucrose, sorbitol, mannitol, starch, acacia gum, calcium phosphate, alginate, gelatin, calcium silicate, microcrystalline cellulose, polyvinylpyrrolidone, cellulose, water (for example, saline or sterile water), syrup, methyl cellulose, methylhydroxybenzoate, propylhydroxybenzoate, talc, magnesium stearate, mineral oil, Ringer's solution, buffer, maltodextrin solution, glycerol, ethanol, or any combination thereof. The pharmaceutical composition may further include a lubricant, a wetting agent, a sweetening agent, a flavoring agent, an emulsifier, a suspending agent, or a preserving agent.

The pharmaceutical composition may be formulated and prepared in the form of a unit dose using a pharmaceutically acceptable carrier and/or diluents, or may be introduced and prepared in a multi-dose container. The pharmaceutical composition may be formulated in a solution of oil or aqueous medium, suspension, syrup, or emulsion. In some aspects, the pharmaceutical composition may be formulated in extracts, powders, powdered drugs, granules, tablets, or capsules. The pharmaceutical composition may further include dispersants or stabilizers. The aqueous medium may contain physiological saline or PBS.

According to another aspect of the present invention, provided is a method of delivering a hydrophobic active ingredient to the target site in the body of a subject comprising: administering a liposome to a subject, wherein the liposome comprises a lipid bilayer, a hydrophobic active ingredient, and an imaging agent to a subject, wherein the hydrophobic active ingredient is packed in the lipid bilayer and the imaging agent is contained in an interior space of the liposome; and applying ultrasound to a target site of the subject to release the hydrophobic active ingredient and the imaging agent from the liposome.

The lipid bilayer, the hydrophobic active ingredient, the imaging agent, and the liposome are as described with respect to the other aspects and methods of the invention.

The subject may be a mammal (e.g., humans).

The administration may be performed by oral administration or parenteral administration. The parenteral administration may comprise, for example, intravenous, intradermal, intramuscular, intracavity (abdominal cavity, joints, or eye) or direct injection. The direct injection may involve injecting directly into a diseased site such as a tumor site. The liposome may be administered intravenously and accordingly brought to the target site such as a tumor site through the circulatory system (i.e., blood flow). The target site may have a leaky property (i.e., have a greater susceptibility to ultrasound/sonoporation due to the target site's physical characteristics). The suitable dosage of the liposome may be prescribed (i.e., determined) according to various factors such as formulation methods, administration methods, patient's age, weight, gender, and morbidity, foods, administration times, administration routes, excretion rates, and reaction sensitivity. Dosage of the liposome may be in a range of about 0.001 mg/kg to about 100 mg/kg.

The hydrophobic active ingredient and the imaging agent may be released simultaneously or sequentially by ultrasound. For example, when the ultrasound has a weak intensity or an exposure time to the ultrasound is short, the imaging agent may be released first. When the ultrasound has a strong intensity or an exposure time to the ultrasound is long, the hydrophobic ingredient may be released first.

The ultrasound may induce sonoporation of the cells at the target site of the subject. Sonoporation refers to the use of ultrasound to increase the permeability of cell membrane or blood vessels. When the permeability of the cell membrane or blood vessels is increased, the absorption of the hydrophobic active ingredients may be increased.

The method may further comprise diagnosing and treating disease by releasing the hydrophobic active ingredient and the imaging agent. The term “diagnosis” as used herein refers to determination of the susceptibility of a subject to certain diseases, determination whether a subject has certain diseases, determination of prognosis of a subject, or therametrics (e.g., monitoring the state of a subject to provide information about therapeutic efficacy). The term “treatment” as used herein refers to the suppression, reduction, or elimination of disease development. Examples of the diseases that may be treated and/or diagnosed include cerebrospinal tumor, head and neck cancer, lung cancer, breast cancer, thymoma, mesothelioma, esophageal cancer, stomach cancer, colon cancer, liver cancer, pancreatic cancer, bile duct cancer, kidney cancer, bladder cancer, prostate cancer, testicular cancer, germ cell tumor, ovarian cancer, uterine cervical cancer, endometrial cancer, lymphoma, acute leukemia, chronic leukemia, multiple myeloma, sarcoma, malignant melanoma, skin cancer, or any combination thereof.

According to embodiments of the present invention, the hydrophobic active ingredient and the imaging agent may be separately introduced into the liposome, and there is no limit to the amount of the hydrophobic active ingredient the liposome may encapsulate. Additionally, the release of the hydrophobic active ingredient and the imaging agent may be controlled, and diagnosis and treatment of diseases may be achieved simultaneously in embodiments of the present invention.

One or more embodiments of the present invention will now be described more fully with reference to the following examples. However, these examples are provided only for illustrative purposes and are not intended to limit the scope of the present invention.

### Example 1

### Preparation of Liposomes Including Hydrophobic Substances and Measurement of Liposome Size

Liposomes in the form of unilamellar vesicles were prepared using 1,2-dipalmitoyl-sn-glycero-3-phosphocholine (DPPC), cholesterol, [1,2-distearoyl-sn-glycero-3-phosphoethanolamine-N-[methoxy(polyethylene glycol)-2000] (ammonium salt)] (DSPE-PEG), SA-V3-NH₂, and sorafenib in a ratio of 55:4:2:1.1 (where a mixing ratio of sorafenib in a main lipid is 3 wt %).

In detail, SA-V3-NH₂ (Peptron, Inc.) was dissolved in ethanol, and DPPC (Avanti Polar lipids, Inc.), cholesterol (Avanti Polar lipids, Inc.), DSPE-PEG (Avanti Polar lipids, Inc.), and sorafenib (Bayer) were dissolved in chloroform. After mixing the ethanol and chloroform solution in a round-bottom flask, a lipid thin layer was formed on the interior wall of the flask by evaporating the solvent at room temperature using a rotary evaporator.

Next, the lipid thin layer was hydrated by adding 150 mM ammonium sulfate solution to the flask at room temperature. The hydrated solution was subjected to vortexing and sonication treatment. Unilamella vesicle type liposomes were prepared by extruding the resulting solution using AVANTI® Mini-Extruder (Avanti Polar Lipids, Inc.) containing a polycarbonate film (Waters Corp.) with pores having a size of 100 nm. A solvent of the prepared liposome solution was passed through a PD-10 (GE Healthcare) desalting column by flowing PBS throughout so that PBS was exchanged with the solvent of the solution.

During the hydration of the lipid membrane, a magnetic resonance (MR) imaging contrast as agent (MULTIHANCE® (Bracco)) was introduced into the liposome interior. A loading process was performed by the ammonium sulfate gradient method, in which a drug was added to the liposome solution formed of liposomes with 250 nM or 150 nM of ammonium sulfate inside and 25 mM of Tris-HCl buffer outside.

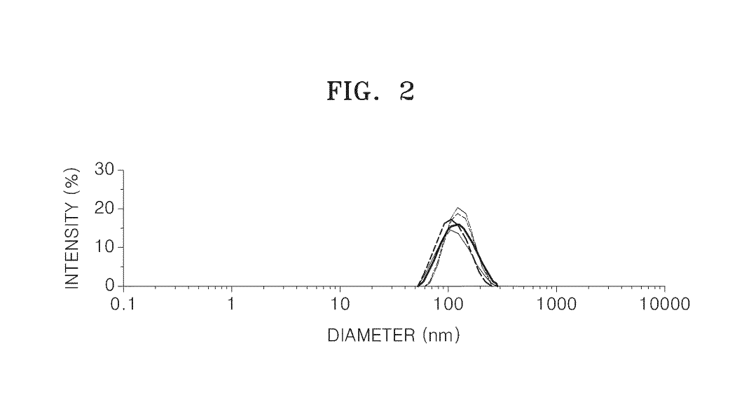

The prepared liposome solution was passed through a PD-10 (GE Healthcare) desalting column by flowing physiological saline throughout to remove any unentrapped drugs. As a result, liposomes including an anticancer drug such as sorafenib entrapped in the lipid bilayer and the MR imaging agent such as MULTIHANCE® entrapped in the aqueous interior were prepared. The sizes of the prepared liposomes were measured using a dynamic light scattering (DSL) analyzer (Malvern Instrument, Ltd.), and the results are shown in FIG. 2. As shown in FIG. 2, the liposomes have a diameter in a range of about 50 nm to about 200 nm, and an average diameter of about 129.3 nm.

### Example 2

### Confirmation of the Form of Liposome Including Hydrophobic Drugs and Imaging Agents

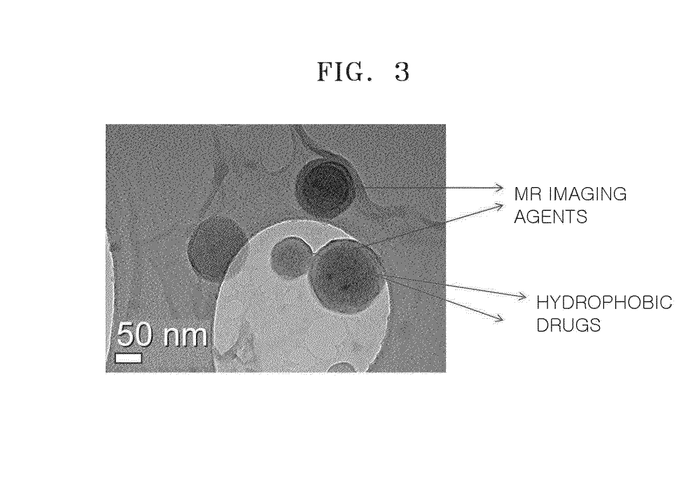

The form of liposomes prepared in Example 1 and thermal-sensitive liposomes containing no doxorubicin were confirmed by a transmission electron microscope (TEM). The liposomes were observed after they were loaded into holey carbon film-supported grids. The grids were then dipped in liquid nitrogen and transferred to a cryotransfer holder (Gatan). A CCD camera (2 k, Gatan) was equipped therewith, and images were obtained using a Tecnai F20 field emission gun electronic microscope that operates at 200 kV (FEI). The obtained images were shown in FIG. 3.

As shown in FIG. 3, spherical liposomes having a diameter in a range of about 100 nm to about 150 nm were confirmed, and the MR imaging agents (MULTIHANCE®) including Gd³⁺ were confirmed in the liposome interior. In addition, the hydrophobic drug (sorafenib) was also confirmed in the liposome membrane.

### Example 3

### Cytotoxicity of Liposomes Including Sorafenib as a Hydrophobic Drug

Cell proliferation was confirmed when the liposome prepared in Example 1 was incubated with cells, followed by ultrasonic treatment.

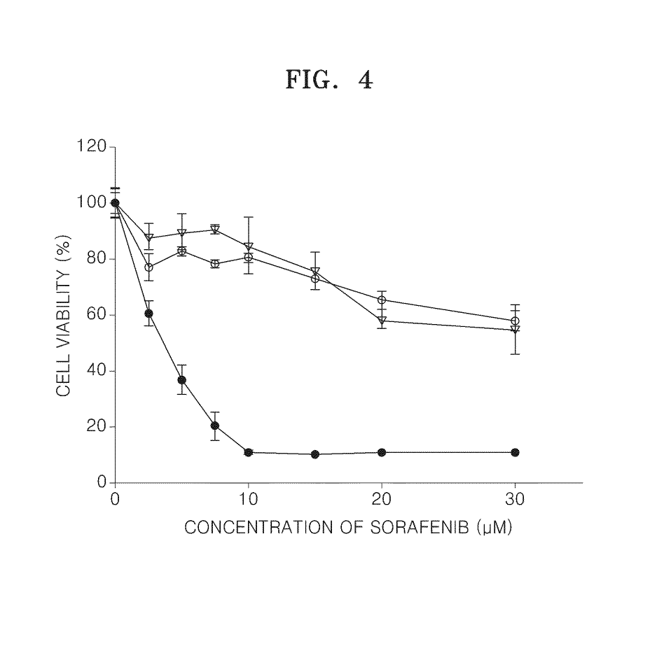

MDA-MB-231 breast cancer cell line (5.0×10⁴ cells) was incubated in Dulbecco's modified Eagle's medium (DMEM) containing 10% (v/v) fetal bovine serum (FBS) and 1% (w/v) penicillin/streptomycin for 24 hours. The cultured MDA-MB-231 cells were treated with the liposomes prepared in Example 1 at a variety of concentrations, immediately placed in a thermoshaker, and then incubated at a temperature of 37° C. for 10 minutes. Next, the MDA-MB-231 cells were subject to ultrasound treatment one time under conditions of a frequency of 20 kHz, a 50% duty cycle, an output of 130 W, and a time for 5 minutes per cycle. Thereafter, the DMEM was replaced with fresh media, and the MDA-MB-231 cells were then cultured at 37° C. for 46 hours. The viability of the cultured MDA-MB-231 cells was measured using WST-8 kit (Dojindo), and the results are shown in FIG. 4 (: sorafenib, ◯: sorafenib-containing liposome, ∇: sorafenib-containing liposome+ultrasound).

As shown in FIG. 4, the cell viability was not affected by a general ultrasound exposure. Accordingly, it was confirmed that sorafenib present in the liposome membrane was not released by a general ultrasound.

### Example 4

### Effects of HIFU on Liposomes Including Hydrophobic Substances

Liposomes were prepared in the same manner as in Example 1, except that 100 μg of the hydrophobic dye Nile Red (Aldrich) was used instead of sorafanib.

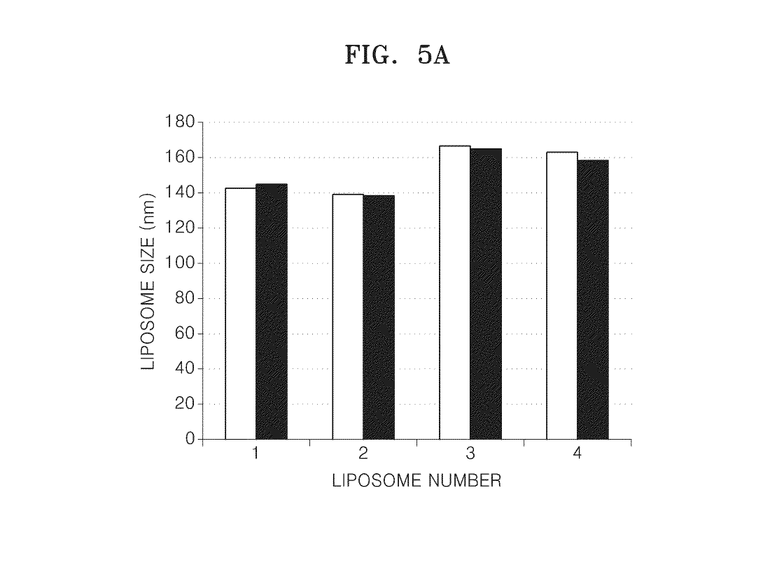

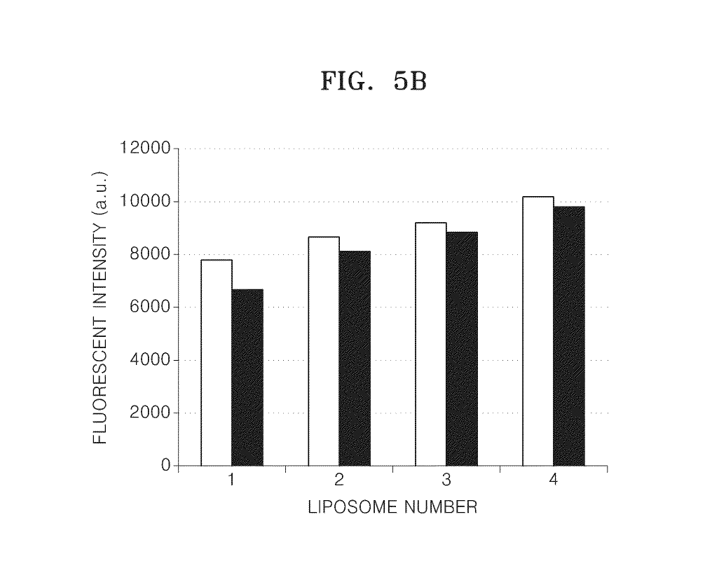

The prepared liposomes were diluted with PBS butter to prepare 4 mg/ml of liposomes. The prepared liposomes were then exposed to HIFU five times under conditions of a frequency of 1.5 MHz, 10% duty cycle, a pulse repetition frequency (PRF) of 1 kHz, an intensity of 40 W, and a time for 4 minutes per cycle. The sizes of the HIFU-exposed liposomes were measured in the same manner as in Example 1, and the results are shown in FIG. 5A (□: before exposure to HIFU, ▪: after exposure to HIFU).

Additionally, the fluorescent intensity of the HIFU-exposed liposomes were measured using a wavelength of 636 nm, and accordingly the amount of Nile Red released was calculated. The results are shown in FIG. 5B (□: before exposure to HIFU, ▪: after exposure to HIFU).

As shown in FIGS. 5A and 5B, the sizes of the liposomes were not significantly different before and after exposure to HIFU. However, it was confirmed that the hydrophobic material such as Nile Red was released by the exposure to HIFU.

### Example 5

### Confirmation of Liposome Compositions to Control Release of Hydrophobic Drugs

This example determined whether the amount of the hydrophobic drugs released is changed according to composition of the phospholipid, which is one of the liposome components.

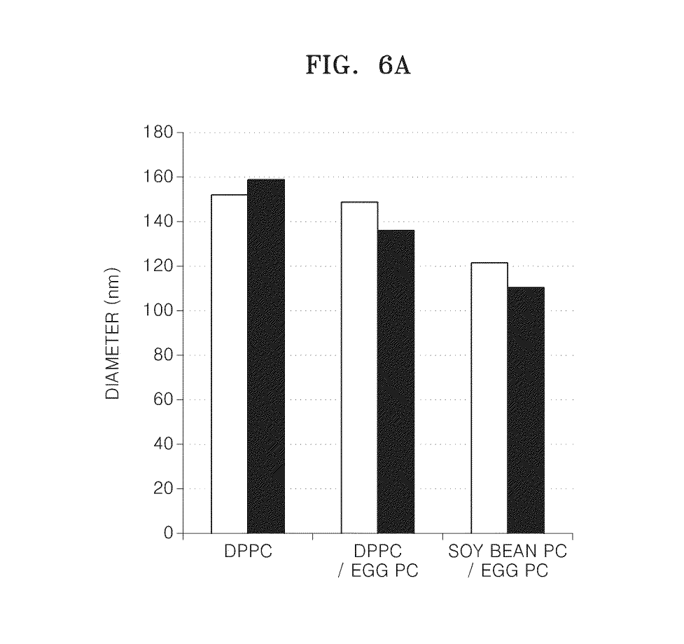

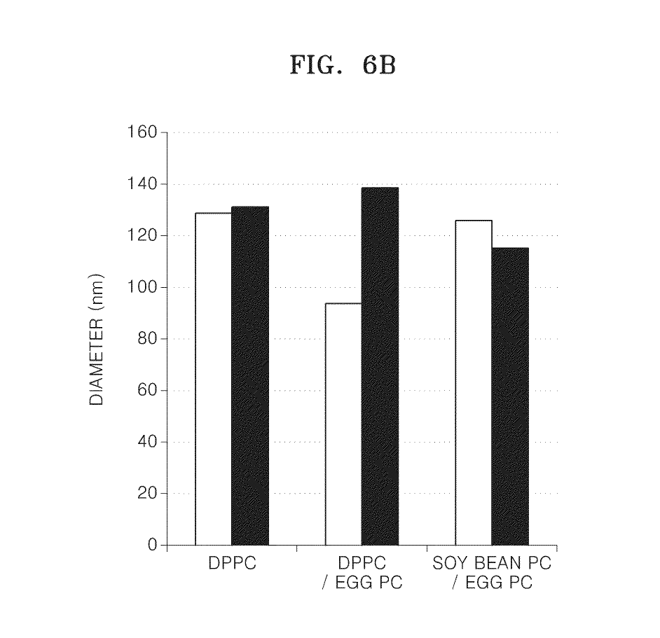

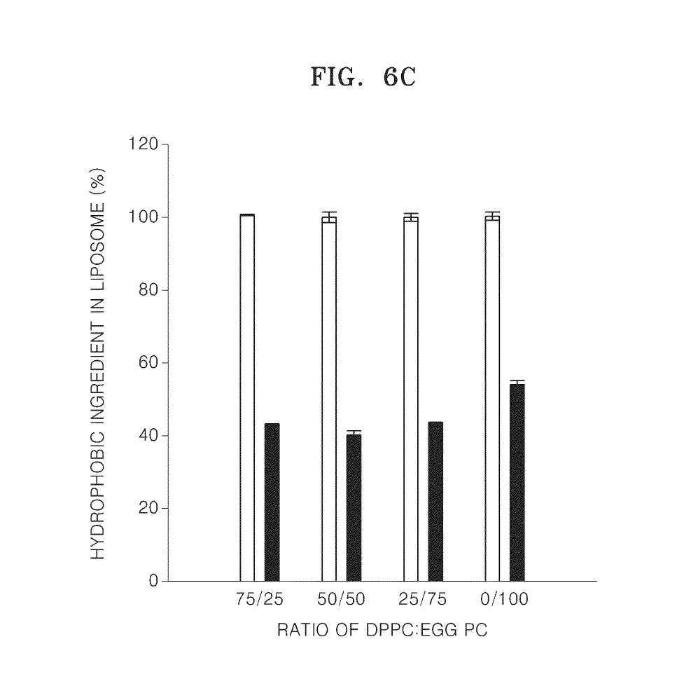

Liposomes were prepared in the same manner as in Example 4 using phospholipids consisting of DPPC only, phospholipids consisting of DPPC and egg PC in a ratio of 1:1, or phospholipids consisting of soy PC and egg PC in a ratio of 1:1. The prepared liposomes were then exposed to HIFU five times under conditions of a frequency of 1.5 MHz, 10% duty cycle, PRF of 1 kHz, intensity of 40 W, and a time for 4 minutes per cycle. As described in Example 1, the sizes of the liposomes were measured before and after exposure to HIFU, and accordingly an average size of the liposomes and a size of 95% or more of liposomes (corresponding to a main peak) were calculated. The results are shown in FIGS. 6A and 6B (□: before exposure to HIFU, ▪: after exposure to HIFU).

As shown in FIGS. 6A and 6B, before and after exposure to HIFU, the difference between the average sizes of the liposomes was within about 10 nm, but the difference between the sizes of 95% or more of liposomes was about 50 nm or greater than about 50 nm. That is, it was confirmed that the phospholipid consisting of a mixed composition had greater effect on liposome size difference than the phospholipid consisting of a single composition.

In addition, the release of the hydrophobic drugs from the liposomes with different ratios of DPPC and egg PC was confirmed before and after exposure to HIFU.

Liposomes consisting of DDPC and egg PC in a ratio of 75:25, 50:50, 25:75, or 0:100 were prepared in the same manner as in Example 4. As described in Example 4, the ratio (%) of the hydrophobic substance present in the liposomes was measured before and after exposure to HIFU, and the results are shown in FIG. 6C (□: before exposure to HIFU, ▪: after exposure to HIFU).

As shown in FIG. 6C, it was confirmed that the liposomes comprising a phospholipid consisting of DPPC and egg PC in a ratio of about 50:50 had released more amounts of the hydrophobic materials than those of that comprised a phospholipid consisting of egg PC only in the liposomes.

### Example 6

### MR Contrast Effects of Liposomes Including Hydrophobic Substances and Imaging Agents

Liposomes including hydrophobic substances such as sorafenib and MULTIHANCE® were prepared in the same manner as in Example 1, and a contrast effect thereof were confirmed.

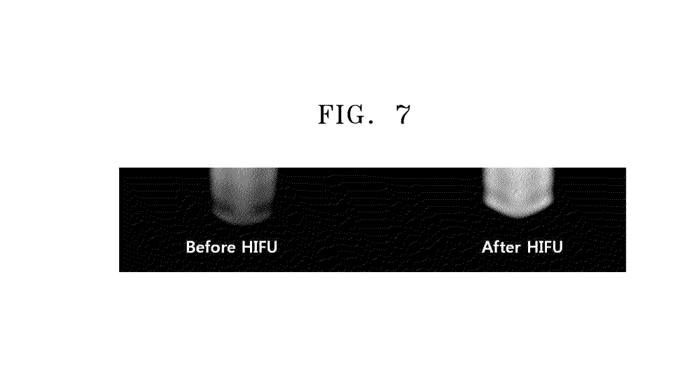

The liposomes prepared in the same manner as in Example 1 were exposed to HIFU five times under conditions of a frequency of 1.5 MHz, 10% duty cycle, PRF of 1 kHz, and a time for 4 minutes per cycle. Thereafter, contrast effects of the liposomes were measured using an MR device 3.0 T Philips Intra Achieva (Philips). The results are shown in FIG. 7.

FIG. 7 is an MRI T1 image showing that the imaging agents entrapped within the liposomes have been released outside after HIFU treatment, and thus the MR contrast image is brightened (that is, an increase of contrast). The MRI brightness is dependent upon the density of hydrogen atoms included in pixels of the image, and is determined by spin-lattice relaxation time (T1) and spin-spin relaxation time (T2) that indicate a time constant of energy exchange between one nuclear spin and another nuclear spin or between one nuclear spin and external lattice, a chemical shift in surroundings, a magnetic susceptibility, temperature, or the like. The MRI imaging agents may alter T1 or T2, which is relaxation time of water molecules in vivo, and accordingly may indirectly contribute to changes in the contrast. The Gd³⁺ (a paramagnetic material changing T1) entrapped within the liposome was released by HIFU stimuli, and shortened the T1 of the water molecules. Accordingly, the T1 of the water molecules outside the liposomes was changed before and after HIFU stimuli, thereby making the contrast higher. As a result, the contrast of the image was increased.

As shown in FIG. 7, the signal intensity of the liposomes were increased about 1.5 times greater after the exposure to HIFU. Therefore, it was confirmed that the imaging agents entrapped within the interior space of the liposomes were released by HIFU stimuli, but the agents entrapped within the interior of the liposomes were not released by temperatures.

As described above, according to the one or more of the above embodiments of the present invention, the use of a liposome including a lipid bilayer, a hydrophobic active ingredient, and a contrast medium, a pharmaceutical composition including the liposome, and a method of delivering an active ingredient to a target site in the body of a subject by using the liposome, may involve the separate introduction of the hydrophobic active ingredient and the contrast medium into the liposome. The amount of the hydrophobic active ingredient to be encapsulated by the liposome is not limited, and the release of the hydrophobic active ingredient and the contrast medium may be controlled. At the same time, diagnosis and treatment of diseases may be achieved.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While one or more embodiments of the present invention have been described with reference to the figures, it will be understood by those of ordinary skill in the art that various changes in form and details may be made therein without departing from the spirit and scope of the present invention as defined by the following claims.

What is claimed is:

1. A liposome comprising:
a lipid bilayer;
a hydrophobic active ingredient; and
an imaging agent;
wherein the hydrophobic active ingredient is in the lipid bilayer and the imaging agent is contained in an interior space of the liposome.

2. The liposome according to claim 1, wherein the liposome is sensitive to one or more types of ultrasound.

3. The liposome according to claim 2, wherein the liposome is sensitive to high intensity focused ultrasound (HIFU).

4. The liposome according to claim 1, wherein the lipid bilayer comprises a phospholipid, a lipid conjugated to polyethylene glycol (PEG), cholesterol, or any combination thereof.

5. The liposome according to claim 4, wherein the liposome comprises a phospholipid, and the phospholipid comprises phosphatidic acid, phosphatidylethanolamine, phosphatidylcholine, phosphatidylserine, phosphatidylinositol, phosphosphingolipid, or any combination thereof.

6. The liposome according to claim 5, wherein the phospholipid comprises 1,2-dipalmitoyl-sn-glycero-3-phosphocholine (DPPC), 1,2-distearoyl-sn-glycero-3-phosphocholine (DSPC), egg phosphatidylcholine, soy bean phosphatidylcholine, or any combination thereof.

7. The liposome according to claim 4, wherein the liposome comprises a phospholipid, and the phospholipid comprises DPPC and egg phosphatidylcholine in a ratio in a range of about 4:1 to about 1:4.

8. The liposome according to claim 1, wherein the hydrophobic active ingredient comprises a hydrophobic drug.

9. The liposome according to claim 8, wherein the hydrophobic drug comprises sorafenib, paclitaxel, cyclosporine A, amphothericin B, indinavir, or any combination thereof.

10. The liposome according to claim 1, wherein the imaging agent comprises a transition element or a chelate complex of the transition element.

11. The liposome according to claim 10, wherein the transition element is Gd³⁺.

12. The liposome according to claim 1, wherein the liposome further comprises a hydrophilic active ingredient.

13. The liposome according to claim 1, wherein the liposome has a diameter of about 50 nm to about 150 nm.

14. A pharmaceutical composition comprising:
a liposome comprising a lipid bilayer, a hydrophobic active ingredient, and an imaging agent, wherein the hydrophobic active ingredient is in the lipid bilayer of the liposome, and the imaging agent is contained in an interior space of the liposome; and
a carrier.

15. A method of delivering a hydrophobic active ingredient to a target site in the body of a subject, the method comprising the steps of:
administering a liposome to a subject, wherein the liposome comprises a lipid bilayer, a hydrophobic active ingredient, and an imaging agent, and the hydrophobic active ingredient is in the lipid bilayer and the imaging agent is contained in an interior space of the liposome; and
applying ultrasound to a target site of the subject to release the hydrophobic ingredient and the imaging agent from the liposome.

16. The method according to claim 15, wherein the ultrasound is HIFU.

17. The method according to claim 16, wherein the ultrasound has a frequency of about 20 kHz to about 2.0 MHz.

18. The method according to claim 15, wherein the hydrophobic active ingredient and the imaging agent are released simultaneously or sequentially.

19. The method according to claim 15, wherein the ultrasound induces sonoporation in the target site of the subject.

20. The method according to claim 15, wherein the method is used for the diagnosis of a disease by releasing the imaging agent from the liposome, and treatment of a disease by releasing the hydrophobic active ingredient from the liposome, wherein the diagnosis and the treatment of the disease are performed simultaneously or sequentially.
